(19) United States
(12) Patent Application Publication
(10) Pub. No.: US 2010/0030110 A1
(43) Pub. Date: Feb. 4, 2010

(54) Glucose meter with er:yag laser lancing device

(76) Inventors: Kee Jung Choi, Daejeon (KR); Hwa Je Woo, Daejeon (KR); Valery G. Polushkin, Moscow Region (RU)

Correspondence Address: STAAS & HALSEY LLP, SUITE 700, 1201 NEW YORK AVENUE, N.W., WASHINGTON, DC, 20005, US

(21) Appl. No.: 11/793,924

(22) PCT Filed: Apr. 21, 2006
(86) PCT No.: PCT/KR06/01511

§ 371 Date: Jun. 22, 2007

(30) Foreign Application Priority Data
Apr. 14, 2006 (KR) 10-2006-0034060

(51) Int. Cl. A61B 5/151

(52) U.S. Cl. 600/583

(57) ABSTRACT

A personal portable painless lancing device and a blood glucose meter using the same, which can lance at the minimum without any pain and use it to measure the blood glucose. The minimum painless lancing device using an Er:YAG laser includes an Er:YAG laser beam-generating section in which the laser beam is generated to punch skin and radiated in a single direction; an adapter installed at the front end of the Er:YAG laser beam-generating section so that the laser beam can be locally concentrated on a skin punch portion; a skin adhesion cap installed at the front end of the adapter so that it can be easily detached, and can reduce the contamination of the Er:YAG laser beam-generating section, which is produced at the time of the lancing, and the danger due to the reuse of it among the patients of lancing; and a case to accommodate the Er:YAG laser beam-generating section and the adapter therein.

### CROSS-REFERENCE TO RELATED APPLICATIONS

This application claims the benefit of Patent Cooperation Treaty Application No. PCT/KR2006/1511, filed Apr. 21, 2006, which claims the benefit of Korean Application No. 10-2006-34060 filed Apr. 14, 2006, the disclosures of which are herein incorporated by reference.

### BACKGROUND

1. Field

Embodiments relate to a painless blood lancing device using the laser and a blood glucose meter using the same, especially, to a new technology for use in the blood glucose monitoring field, which is capable of carrying out the lancing and blood glucose measuring by means of a single equipment. More particularly, the present invention relates to a personal portable painless lancing device, which is capable of lancing from the skin without any pain by using an Er:YAG laser technology in the medical field, and further by using a laser beam having a wavelength of 2.94 μm, which has a strong absorption force with respect to water so that it does not harm the human body, and a blood glucose meter using the same.

2. Description of the Related Art

In general, an Er:YAG laser beam-generating device (laser head) used to lance without any pain includes a part called a reflector in the outer shape, which is made by depositing or plating Ag/Ni/Cu or alloy thereof on a surface of a metal body several times and machining the surface thereof, and a flash lamp to pump the laser beam and an Er:YAG crystal are built in the reflector. The strength of the laser beam radiated from the Er:YAG laser beam-generating device varies based on the radiation amount of the flash lamp and the volume, size and the density of the Er:YAG crystal.

However, the conventional painless lancing device using a laser was not good at adhesion property between the skin and the Er:YAG laser beam-generating device, so that there occurred problems that sound such as “flump” was produced when the skin was punched by the laser beam, a white spot was produced at the skin from which the blood had been collected, and smoke produced from the burned skin was blown out to thereby make the mind of the lanced patient uncomfortable. Also, the retina can be injured due to strong stimulation, because a flint of the flash lamp and the laser beam radiated at the skin were in view.

Thus, the conventional painless lancing device using the Er:YAG laser beam-generating section resulted in the shrinkage of the intention for use it due to the fear complex about the lancing, despite the advantage that it could lance without any pain in contrast to a needle shaped lancet.

Reviewing the technical field of art using a strip to measure the blood glucose, three major companies such as Johnson & Johnson, Abbot in the US and Roche of Deutschland have commanded the market in the world, and Allmedicus, Infopia, i-sense, and the like in Korea have put the equipment to measure the blood glucose after lancing by means of the needle shaped lancet in the market.

In case of an insulin-dependent patient who should check the blood glucose in real time for two or three times a day, the lancing is usually carried out by piercing the end portion of a finger by means of a needle shaped lancet. However, this lancing method causes big pains in addition to the lancing beyond the necessary amount of blood, because the lancing is carried out by the incision of the skin tissue by means of the lancet. Moreover, while it is necessary to stop bleeding of a portion from which the blood has been collected, the incision portion will not be cured quickly after the stop of the bleeding, so that there is existed the possibility of infection due to virus or germs.

Furthermore, while there has been recently disclosed an infrared-ray scanning type blood glucose meter to indirectly measure the blood glucose, it is not widely used because the reliability of the measured value is relatively degraded as compared to the direct lancing method.

Meanwhile, it was inconvenient to purchase the equipment and maintain it because the lancing device and the blood glucose meter were separately manufactured and provided conventionally so that the measuring of the blood glucose could be performed only after the both equipment are all prepared.

### SUMMARY

Additional aspects and/or advantages will be set forth in part in the description which follows and, in part, will be apparent from the description, or may be learned by practice of the invention.

Accordingly, the present invention was devised to solve such conventional problems, and it is an aspect of the present invention to provide a painless laser lancing device, which is not exposed to flint or smoke produced from the burning of the skin as the adhesion property between the skin and the Er:YAG laser beam-generating section is excellent, the lancing amount for one time is very small, and no pain was caused in lancing.

An aspect of the present invention is to provide a personal portable blood glucose meter made by incorporating the painless lancing device and the blood glucose measuring device into a unitary equipment.

To achieve the above aspects/other aspects, according to embodiments of the present invention, the painless minimum lancing device to collect the blood by using an Er:YAG laser concentrates the laser beam on the skin locally and enhances the adhesion property between the skin and the laser beam-generating section to thereby overcome the psychological rejection symptom of the lanced patient.

In this regard, the painless minimum lancing device to collect the blood by using an Er:YAG laser according to embodiments of the present invention is characterized by including an Er:YAG laser beam-generating section to generate the laser beam required to punch skin; an adapter mounted at the front end of the Er:YAG laser beam-generating section so that the laser beam can be concentrated on the skin locally; and a case to accommodate the Er:YAG laser beam-generating section and the adapter.

As described above, embodiments according to the present invention can prevent the infection from the virus or the germs without separate sterilization of the lancing portion, since the radiated laser beam instantly heats the skin to above about 1000° C. to thereby perform the sterilization.

Furthermore, it is possible to primarily remove the smell and smoke produced from the burning of the skin and the occurrence of dull noise by disposing the adapter and the skin adhesion cap having good adhesion property with the skin at the front end of the Er:YAG laser beam-generating section.

Also, it is possible to intercept the exposure of the flash to the outside by means of the case made of engineering plastic.

### BRIEF DESCRIPTION OF THE DRAWINGS

These and/or other aspects and advantages will become apparent and more readily appreciated from the following description of the embodiments, taken in conjunction with the accompanying drawings of which:

FIG. 1 illustrates the basic structure of a painless minimum lancing device using an Er:YAG laser according to embodiments of the present invention;

FIG. 2 is a model view showing a minimum painless lancing device using an Er:YAG laser according to an embodiment of the present invention;

FIG. 3 is a model view showing a skin adhesion cap used in an embodiment of the present invention;

FIG. 4 is a solid model view showing the skin adhesion cap of FIG. 3 taken from the rear side;

FIG. 5 is an exploded perspective view showing an example of accommodating an adapter and an interlock switch at one side of the case according to an embodiment of the present invention;

FIG. 6 is a view showing an inner status of the painless minimum lancing device using an Er:YAG laser according to an embodiment of the present invention, by opening one side of the case;

FIG. 7 is a view showing an inner construction of the painless lancing device by removing a part of the case material at a connection portion between the skin adhesion cap shown in FIG. 6 and the Er:YAG laser beam-generating section;

FIG. 8 is a construction view showing a blood glucose meter according to an embodiment of the present invention.

### DETAILED DESCRIPTION OF THE EMBODIMENTS

### EXPLANATION OF THE DRAWING NUMERALS

1: front surface securing cover
2: reflector
3: flash lamp
4: rear surface securing cover
5: focusing lens
6: focusing lens holder
7: semi-permeable mirror
8: front surface securing holder
9: Er:YAG crystal
10: rear surface securing holder
11: total reflection mirror
12: inspection film
13: skin adhesion cap
14: connection device
15: buffer spring
16: support jaw
21: capacitor
22: charging transformer
23: blood glucose measuring circuit
24: blood glucose sensor strip
25: connector
26: input and output device
27: triggering transformer
28: laser beam control circuit
29: electric power supply source (charging battery)
30: MPU and peripheral circuit
31: interlock switch operating handle
32: case
33: interlock switch securing holder

The painless minimum lancing device to settle the technical task according to embodiments of the present invention is characterized by comprising an Er:YAG laser beam-generating section to generate a laser beam required to punch skin; an adapter installed at the front end of the Er:YAG laser beam-generating section so that the laser beam can be locally concentrated on a skin punch portion; and a case to accommodate the Er:YAG laser beam-generating section and the adapter therein.

More particularly, according to an aspect of the present invention, the lancing device can include an Er:YAG laser beam-generating section from which the laser beam (beam) is generated and radiated in a single direction; an adapter installed at the front end of the Er:YAG laser beam-generating section, and disposed in an axis identical with that of an Er:YAG crystal with a predetermined distance from the Er:YAG crystal constructing the Er:YAG laser beam-generating section; a skin adhesion cap installed at the front end of the adapter so that it can be in close contact with the skin, and is capable of preventing the contamination of the Er:YAG laser beam-generating section due to the blood gushed out at the time of the skin punch and the smoke produced from the burning of the skin, and confirming if it is reused by the naked eye; and a case to accommodate the Er:YAG laser beam-generating section and the adapter therein.

Also, according to an aspect of the present invention there can be included a body penetratingly formed internally to make the laser beam pass through, secured detachably to the front end of the adapter at a rear end portion thereof, and inclined by 68 at a front end thereof with respect to the laser beam passing through the inside of the body so that it is in close contact with the skin; and an inspection film installed at the rear end of the body to enable inspection if the skin adhesion cap is used, to prevent the dispersion of the blood occurring at the time of the lancing and the contamination of the adapter due to smoke, and to remove the problem of infection of the patient due to reuse of the skin adhesion cap.

In this instance, the skin adhesion cap can be made from polyester, which is hot compressed with high strength, and the like, so that noise/flash light/smoke and the like arising at the time of the laser beam radiation can be intercepted, and is made in structure such that the skin can be a little pushed and entered into the front end of the body, when it is contacted with a lancing portion. Also, in respect of sanitation, the skin adhesion cap is constructed to be easily detachable from the front end of the adapter so that it can be disposable. In addition, more preferably, a support jaw can be formed at the outer surface of the body of the skin adhesion cap to prevent the disturbance with intercepting the passing through of the air when it is used.

In addition, the Er:YAG laser beam-generating section may include a reflector having a receiving space formed at the inside;

an Er:YAG crystal disposed in the receiving space of the reflector;

a total reflection mirror installed at the rear surface of the Er:YAG crystal;

a semi-permeable mirror installed at the front end of the Er:YAG crystal;

a flash lamp installed at the inside of the receiving space in such a manner as to be spaced apart by a certain interval from the Er:YAG crystal;

front and rear surface securing covers respectively installed at both front and rear ends of the reflector so that both ends of the Er:YAG crystal and the flash lamp can be supported; and

a front and rear surface securing holder inserted into the outside of both sides of the front and rear ends of the reflector for integrally securing the front and rear surface securing cover and the reflector.

Moreover, the adapter may preferably include a focusing lens holder inserted into the front surface securing holder of the Er:YAG laser beam-generating section;

a focusing lens inserted into an inside passage of the focusing lens holder so that the laser beam can be concentrated on a skin punch portion to enable the painless lancing;

a connection device for the skin adhesion cap, which is installed at the front end of the focusing lens holder; and

a buffer spring interposed between the connection device and the focusing lens holder.

According to the construction for embodiments of the invention as described above, it is possible to prevent the contamination of the adapter and the focusing lens due to blood and smoke gushed out at the time of the lancing, by means of the contamination prevention function of the skin adhesion cap detachably mounted to the connection device, and it is possible to radically reduce the psychological pain and the real pain of the lanced patient because the skin injury of the lancing portion can be minimized. Also, the spring inserted between the adapter and the focusing lens holder performs buffer action when the disposable skin adhesion cap is contacted with the skin.

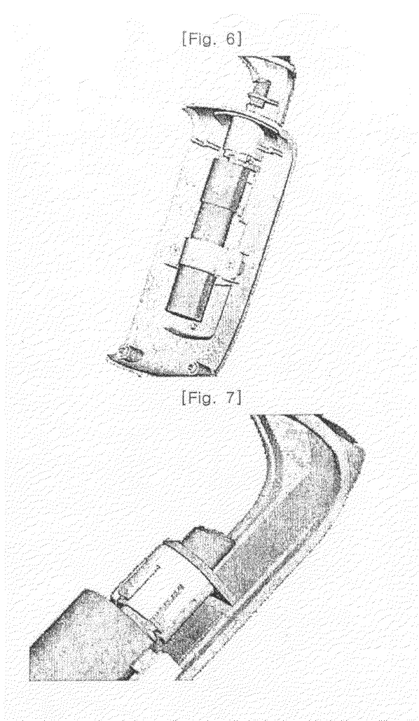

Moreover, with regard to the painless minimum lancing device using the Er:YAG laser according to embodiments of the present invention, as shown in FIGS. 5 through 7, since an interlock switch is mounted at the case to which the adapter is accommodated, the laser beam cannot be used for other object or use than the lancing.

Furthermore, another aspect of the present invention, the blood glucose meter includes, in addition to the painless minimum lancing device using an Er:YAG laser as described above, a connector to insert a blood glucose sensor strip, a blood glucose measuring circuit, and means to display measured blood glucose value including LCD, and the like, so that the lancing and the blood glucose measuring can be performed with a single equipment. Also, it is preferable that a microprocessor is loaded on a PCB to control the operation of the respective constructional element, and a program to record/manage past results of the blood glucose measuring is built therein.

In addition, the painless minimum lancing device using an Er:YAG laser and the blood glucose meter using the same require an input and output means to display the operation/management and status/result, and it is very easy for those skilled in the field of art to conceive it.

In other words, it has been well known in the field of art to which the present invention pertains, and in the field of electronic industry to construct the control method and device for the output energy of the laser beam, the control method and the device to focus of the laser beam, means to display the status/result, and the like, within the scope of the technical spirit of the present invention. Thus, detailed referring and description of the input and output means are abridged in the present specification.

Hereinafter, the construction and action of the painless minimum lancing device using an Er:YAG laser and a blood glucose meter according to an embodiment of the present invention will be described in detail with reference to the appended drawings.

### 1. A Painless Minimum Lancing Device Using an Er:YAG Laser

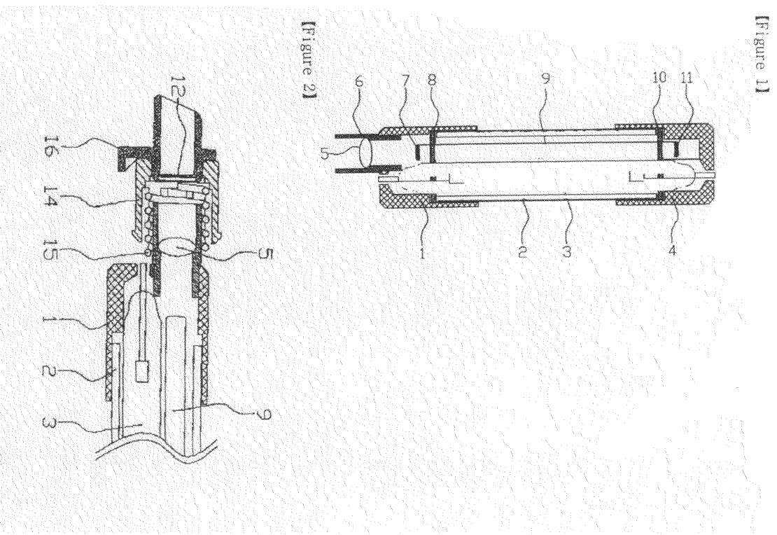

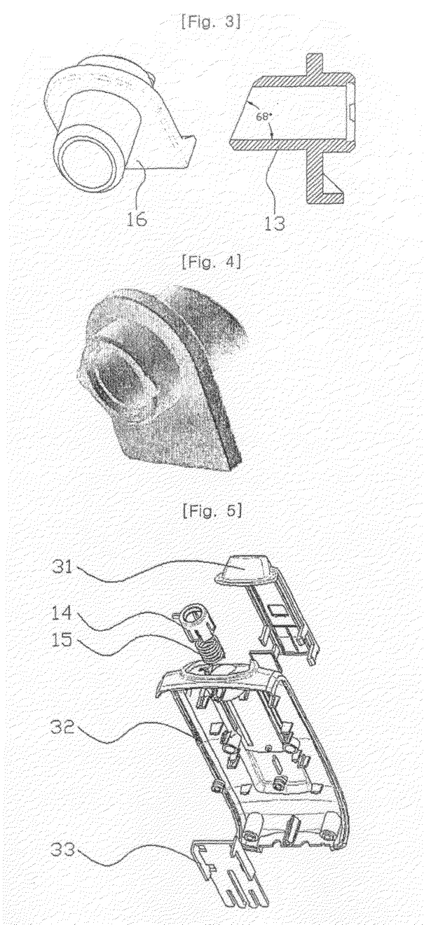

FIGS. 1 through 4 illustrate a painless lancing device using a laser according to an embodiment of the present invention, an adapter becoming one element of the primary constructional elements, and a disposable skin adhesion cap.

As shown in FIG. 1, an Er:YAG laser beam-generating section of the present embodiment includes a reflector 2 having a receiving space formed at the inside, an Er:YAG crystal 9 inserted into the receiving space of the reflector 2 so that both ends thereof are exposed to the outside of the receiving space, mirrors 7, 11 secured at both ends of the Er:YAG crystal 9, and a flash lamp 3 inserted into the receiving space of the reflector with maintaining a predetermined distance from the Er:YAG crystal 9. Both ends of the Er:YAG crystal and the flash lamp are supported by a front surface securing cover 1 and a rear surface securing cover 4 installed respectively at both ends of the reflector so that they cannot move, and front and rear surface securing holders 8, 10 are respectively inserted outwardly at front and rear both ends of the reflector for integrally securing the front and rear surface securing covers and the reflector.

Herein, an semi-permeable mirror 7 is installed at the front of the Er:YAG crystal 9, and a total reflection mirror 11 is installed at the rear side.

The Er:YAG crystal 9 functions to produce a laser beam strong to hydrophile property and having a wavelength of 2.94 μm of 0.1˜0.6 mJ by means of lasing transition 4I11/2→4I13/2 of the crystal after inducing and focusing flash radiated from the flash lamp 3, and the strength of the laser beam is determined based on the crystallization structure, density, volume of the crystal, and the light emitting amount of the flash lamp.

An embodiment of the present embodiment, the crystal is made by forming by doping fine Yttrium Aluminum Garnet (Y₃A₁₅G₁₂) powder at the melting temperature of 1870° C. so that the laser beam can be radiated uniformly in a predetermined direction and concurrently the strength can be highly maintained, performing surface treatment with OH-free special coating, and then precisely machining the resultant product with a surface roughness below 1 μm.

Herein, since the Er:YAG crystal 9 should be maintained at a clear and transparent status in the crystallization structure, it takes substantially long time in doping, and it requires know-how of the skilled technical manpower because minute shear machining and grinding machining below 1 μm are required to maintain the linearity of the laser beam (beam) and to remove interference and scattering.

The flash lamp 3 to pump the laser beam is a device to produce the flash of about 80˜100 WS so as to supply energy to the Er:YAG crystal 9. In the present embodiment, it is induced to discharge the flash of about 80˜100 WS level by using the trigger voltage, after filling a cylindrical stick type glass tube under the vacuum state with xenon gas of 400˜700 mmHg and applying a high voltage at both electrodes installed with an interval of about 40˜45 mm there-between.

The reflector made by installing the Er:YAG crystal 9 and the flash lamp 3 with a predetermined interval is an element to form outer appearance of the Er:YAG laser beam-generating section, and functions to induce and focus the flash light, which is emitted from the flash lamp 3 and reflected at the inner surface of the reflector not less than 98%, to the Er:YAG crystal 9.

According to an aspect of the present invention, primarily silver is vapor deposited on the surface of the quartz glass to thereby maintain the reflection ratio not less than 98% at the time of manufacturing the reflector, and performing the coating of an alloy made of nickel and copper to prevent the oxidation of the silver (Ag), and then performing the milling of the surface thereof.

The mirrors denoted by the numerals 7 and 11 are elements to make the laser beam focused by the Er:YAG crystal 9 be radiated with maintaining the linearity in the predetermined direction, then the mirrors are machined minutely so that the flat degree of 10-9 m can be maintained according to the radiation angle and the direction of the laser beam, and the surface of them are made smooth by the polishing.

Also, the semi-permeable mirror 7 installed at the direction (front end) in which the laser beam is radiated, is machined so that permeation ratio can be maintained above 50%, and the total reflection mirror 11 installed at the opposing side (rear side) is machined so that the laser beam focused to the Er:YAG crystal can be reflected in the direction in which they are radiated not less than 90% to thereby maintain the linearity and contrive to induce the radiating direction.

This is achieved by improving the conventional Er:YAG laser technology, and resulted in the minimization of the laser beam loss due to interference and scattering and improvement in the permeation degree and the linearity of the laser beam by installing the total reflection/semi-permeable mirrors 7, 11 at both ends of the Er:YAG laser beam-generating section respectively.

As shown in FIGS. 1 and 2, the adapter of the embodiment described for the present invention is installed at the front side of the total reflection mirror 7 of the Er:YAG laser beam-generating section, for example.

Also, the Er:YAG laser beam-generating section includes a focusing lens holder 6, a focusing lens 5 inserted into an inner passage of the focusing lens holder, a connection device 14 inserted into an outer surface of the focusing lens holder, and a buffer spring 15 interposed between them.

The focusing lens 5 is a construction element to focus the laser beam radiated in the predetermined direction at the skin to lance with an accurate focus, and the skin penetration depth is made to be not more than 0.5 mm and the diameter is in the range of 0.3 mm lest it should stimulate a nervous cell in the inner skin tissue to thereby cause pains, although it can punch a hole of 0.1˜0.5 mm diameter to a depth of 0.2˜4 mm. In order to maintain the skin penetration depth and the punch diameter, the distance from the total reflection mirror 7 in the direction to which the laser beam is radiated to the focusing lens is maintained to be in the range of 13˜15 mm, and the distance from the focusing lens to the skin surface is adjusted to be in the range of 17.5˜18.0 mm.

As a result, it is possible to easily collect the blood not less than 0.5 μl required to measure the blood glucose by stimulating the blood flow of the capillary existing between the outer tissue and the inner tissue to be in the range of 400 μsec˜500 μsec, and minimize the size of the injury portion and the pain.

It is possible to adjust the lancing amount to be 1 μl˜500 μl according to the energy strength of the laser beam, and there is an advantage of primary preventing the occurrence of the smell produced from the burning of the skin and dull noise of “flump” at the time of the lancing. Also, according to the present invention, the energy loss due to the interference and scattering is minimized and the efficiency has been improved by performing the polishing to 10⁻⁹ m after minutely machining the surface of the focusing lens 5.

The adapter of the present embodiment as described above functions to intercept/absorb the flash light produced from the flash lamp and the noise produced from the lancing, which were the basic problems of the prior art.

In other words, according to embodiments of the present invention, the adaptor can be a kind of “airtight explosion-proof type” adapter.

In addition, the connection device 14 inserted into the front end of the focusing lens holder having a focusing lens built therein is made from compression polyester material to improve the effect of the noise interception through maintaining the airtight property.

Moreover, the periphery of it is sealed by means of a thermal contraction tube made of foamed compression type engineering plastic material to intercept the flash light radiated from the flash lamp 3 and the final noise.

The skin adhesion cap denoted by the drawing numeral 13 is constructed to be easily inserted into the connection device 14 at the front end of the adapter when it is pressed by the finger.

The skin adhesion cap may be made of flexible soft material (for instance, silicon rubber) so that it can be contacted with a little compression state to maintain airtight property so as to easily collect the blood and remove the pain caused by the lancing.

Also, since the skin adhesion cap of the present embodiment is provided with a thin inspection film 12 made of polyethylene material at the rear cylindrical end thereof, which is inserted into the inside of the connection device 14 of the adapter and met with the focusing lens, it is possible to prevent the decrease of the linearity and the permeation ratio of the laser beam at the time of the punch of the skin with the Er:YAG laser beam due to the staining of the blood at the focusing lens, and prevent the contamination of the collected blood. In addition, since the polyethylene film is punched by the laser beam at the time of radiating the laser beam (that is, while it is used at least once), it is possible to simply confirm with the naked eye whether it can be used again. Moreover, a support jaw 16 is projected from the outer surface of the body of the skin adhesion cap to prevent the swing at the time of the lancing.

Furthermore, the painless minimum lancing device using the Er:YAG laser according to the present embodiment is provided with two safety devices.

One safety device of the painless minimum lancing device is an interlock sensor (not shown in the drawing) installed at the connection device to continuously maintain the locking state unless the skin adhesion cap 13 is completely secured at the connection device 14 of the adapter. Thus, the interlock sensor is operated to radiate the laser beam only when the finger is pressed with being contacted with the skin adhesion cap to thereby be completely secured to the connection device.

The other is, as shown in FIG. 5, a construction in which the interlock sensor (not shown) is secured at an interlock switch securing holder 33 disposed at the inside of the case 32, and a stopper shaped handle 31 to operate the interlock switch, which is disposed at the outside of the case, is operated to radiate the laser beam. That is, these safety devices function so that the laser beam cannot be radiated on other portions of the human body than the finger.

### 2. Blood Glucose Meter

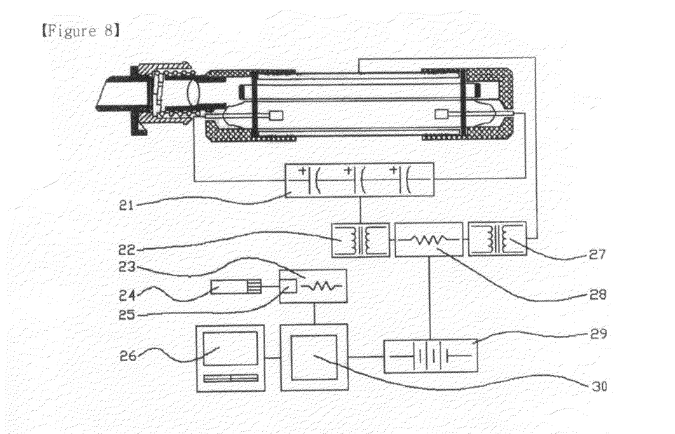

FIG. 8 illustrates a blood glucose meter according to an embodiment of the present invention.

As shown in the drawing, the blood glucose meter is constructed by including total reflection/semi-permeable mirrors 7, 11 respectively installed at both ends of an Er:YAG crystal 9, and a PCB embodied by a laser beam control circuit 28 consisted of a high output capacitor 21 and minimal charging/triggering transformers 22, 27, so that it can be used at any time regardless of the place, to thereby achieve the miniaturization and the light-weight of the device.

Also, the blood glucose meter employs a power supply device 29 using a lithium-ion battery to enable the lancing and the blood glucose measuring above one hundred times without the supply of the AC electric power, and a peripheral circuit 30 including a microprocessor unit (MPU) is embodied into a small PCB.

The laser beam control circuit 28 embodied into a PCB charges voltage applied from the transformer to the capacitor and controls the amount of the electric power source by means of the control circuit, to thereby adjust the emitting amount of the flash lamp to be 80˜100 WS, resulting in the control of the strength of the Er:YAG laser beam to be from 0.1 J in the minimum to 0.6 J in the maximum.

In addition, the safety device to intercept the laser beam from being radiated on a portion except the lancing portion, is coupled in movement to the display of [READY] of the input and output device 26 including the laser beam control circuit 28 and the LCD, so that it can be used only for the lancing and the blood glucose measuring of the finger.

The connector denoted by the drawing numeral 25 is a construction element to insert a blood glucose sensor strip 24 with blood glucose information stored therein, and the blood glucose information stored in the blood glucose sensor strip is transferred to a blood glucose measuring circuit 23, which is embodied into a PCB, and displayed as numerical value through the LCD, and the measured blood glucose value is stored.

Herein, the blood glucose sensor strip is a strip to collect the blood of 0.5 μl by means of the method using the Er:YAG laser and providing the blood glucose information according to the electric chemical reactions, and comprises a blood insert absorbing the collected blood, an insert sensor to determine if it is possible to collect the blood glucose information from the absorbed blood, and a contact type connector 25 to transfer the collected blood glucose information to the blood glucose measuring circuit 23 by means of the electric chemical analysis method after reacting it with a reagent injected on the strip.

Accordingly, it is possible to accurately measure the blood glucose with minimum blood.

In the present embodiment, a 32 bit MPU and a peripheral circuit are installed on the PCB and an operation software is built therein so as to control/perform the blood glucose measuring as described above, to thereby achieve the simplification and the miniaturization of the constructing circuit, so that it is possible to prevent the causing factors of the measuring error, occurrence of several disorders and erroneous action due to the complex circuit construction in advance.

Meanwhile, the blood glucose meter according to embodiments of the present invention includes a control button in addition to the construction elements as described above, to vary the strength of the laser beam from eight stages in the minimum to sixteen stages in the maximum, by controlling the amount of the electricity supplied to the flash lamp according to the feature of the lancing people, by means of the voltage from 400V in the minimum to 700V in the maximum applied to an anode and a cathode.

Also, the blood glucose meter of the present embodiment has a desired software program installed therein to display the past blood glucose measuring data, remaining amount of the charging battery, and date and time of the blood glucose value, and the like, by means of numeric values or symbols in the LCD display.

As described above, embodiments of the present invention collect the blood of about 0.5 μl without any pain and use it in the blood glucose measuring, so that it will be very advantageous if the present device is supplied to the insulin-dependent patient (a diabetic).

Furthermore, embodiments of the present invention can prevent the infection from the virus or the germs without separate sterilization of the lancing portion, since the radiated laser beam instantly heats the skin to above about 1000° C. to thereby perform the sterilization.

Also, it is possible to primarily remove the smell and smoke from the burning of the skin and the occurrence of dull noise by disposing the adapter and the skin adhesion cap having good adhesion property with the skin at the front end of the Er:YAG laser beam-generating section.

Moreover, it is possible to intercept the exposure of the flash light to the outside by means of the case made of an engineering plastic.

Although a few embodiments have been shown and described, it would be appreciated by those skilled in the art that changes may be made in these embodiments without departing from the principles and spirit of the invention, the scope of which is defined in the claims and their equivalents.

What is claimed is:

1. A painless minimum lancing device using an Er:YAG laser, comprising:
an Er:YAG laser beam-generating section to generate a laser beam required to punch skin;
an adapter installed at the front end of the Er:YAG laser beam-generating section so that the laser beam can be locally concentrated on a skin punch portion; and
a case to accommodate the Er:YAG laser beam-generating section and the adapter therein.

2. The painless minimum lancing device according to claim 1, further comprising a skin adhesion cap detachably installed at the front end of the adapter

3. The painless minimum lancing device according to claim 2, wherein the skin adhesion cap includes:
a body penetratingly formed internally so that the laser beam can pass through, secured detachably to the front end of the adapter at a rear end portion thereof, and inclined at a front end thereof so that it can be in close contact with the skin; and
an inspection film installed at the rear end of the body so as to inspect if the skin adhesion cap is used, and to prevent the dispersion of the blood occurring at the time of the lancing and the contamination of the adapter due to the smoke, and to remove the problem of infection of the patient due to reuse of the skin adhesion cap.

4. The painless minimum lancing device according to claim 3, wherein the front end portion of the body of the skin adhesion cap is inclined by 68° with respect to the laser beam passing through the inside of the body.

5. The painless minimum lancing device according to claim 1, wherein the Er:YAG laser beam-generating section includes:
a reflector having a receiving space formed at the inside;
an Er:YAG crystal disposed in the receiving space of the reflector;
a total reflection mirror installed at the rear surface of the Er:YAG crystal;
a semi-permeable mirror installed at the front end of the Er:YAG crystal;
a flash lamp installed at the inside of the receiving space in such a manner as to be spaced apart by a certain interval from the Er:YAG crystal;
front and rear surface securing covers respectively installed at both front and rear ends of the reflector so that both ends of the Er:YAG crystal and the flash lamp can be supported; and
a front and rear surface securing holder, inserted into the outside of both sides of the front and rear ends of the reflector, to integrally secure the front and rear surface securing cover and the reflector.

6. The painless minimum lancing device according to claim 2, wherein the Er:YAG laser beam-generating section includes:
a reflector having a receiving space formed at the inside;
an Er:YAG crystal disposed in the receiving space of the reflector;
a total reflection mirror installed at the rear surface of the Er:YAG crystal;
a semi-permeable mirror installed at the front end of the Er:YAG crystal;
a flash lamp installed at the inside of the receiving space in such a manner as to be spaced apart by a certain interval from the Er:YAG crystal;
front and rear surface securing covers respectively installed at both front and rear ends of the reflector so that both ends of the Er:YAG crystal and the flash lamp can be supported; and
a front and rear surface securing holder, inserted into the outside of both sides of the front and rear ends of the reflector, to integrally secure the front and rear surface securing cover and the reflector.

7. The painless minimum lancing device according to claim 3, wherein the Er:YAG laser beam-generating section includes:
a reflector having a receiving space formed at the inside;
an Er:YAG crystal disposed in the receiving space of the reflector;
a total reflection mirror installed at the rear surface of the Er:YAG crystal;
a semi-permeable mirror installed at the front end of the Er:YAG crystal;
a flash lamp installed at the inside of the receiving space in such a manner as to be spaced apart by a certain interval from the Er:YAG crystal;
front and rear surface securing covers respectively installed at both front and rear ends of the reflector so that both ends of the Er:YAG crystal and the flash lamp can be supported; and
a front and rear surface securing holder, inserted into the outside of both sides of the front and rear ends of the reflector, to integrally secure the front and rear surface securing cover and the reflector.

8. The painless minimum lancing device according to claim 4, wherein the Er:YAG laser beam-generating section includes:
a reflector having a receiving space formed at the inside;
an Er:YAG crystal disposed in the receiving space of the reflector;
a total reflection mirror installed at the rear surface of the Er:YAG crystal;
a semi-permeable mirror installed at the front end of the Er:YAG crystal;
a flash lamp installed at the inside of the receiving space in such a manner as to be spaced apart by a certain interval from the Er:YAG crystal;
front and rear surface securing covers respectively installed at both front and rear ends of the reflector so that both ends of the Er:YAG crystal and the flash lamp can be supported; and
a front and rear surface securing holder, inserted into the outside of both sides of the front and rear ends of the reflector, to integrally secure the front and rear surface securing cover and the reflector.

9. The painless minimum lancing device according to claim 1, wherein the adapter includes:
a focusing lens holder inserted at one end thereof into the front surface securing holder of the Er:YAG laser beam-generating section;
a focusing lens inserted into an inside passage of the focusing lens holder so that the laser beam can be concentrated on a skin punch portion;
a connection device installed at the front end of the focusing lens holder; and
a buffer spring interposed between the connection device and the focusing lens holder.

10. The painless minimum lancing device according to claim 2, wherein the adapter includes:
a focusing lens holder inserted at one end thereof into the front surface securing holder of the Er:YAG laser beam-generating section;
a focusing lens inserted into an inside passage of the focusing lens holder so that the laser beam can be concentrated on a skin punch portion;
a connection device installed at the front end of the focusing lens holder; and
a buffer spring interposed between the connection device and the focusing lens holder.

11. The painless minimum lancing device according to claim 3, wherein the adapter includes:
a focusing lens holder inserted at one end thereof into the front surface securing holder of the Er:YAG laser beam-generating section;
a focusing lens inserted into an inside passage of the focusing lens holder so that the laser beam can be concentrated on a skin punch portion;
a connection device installed at the front end of the focusing lens holder; and
a buffer spring interposed between the connection device and the focusing lens holder.

12. The painless minimum lancing device according to claim 4, wherein the adapter includes:
a focusing lens holder inserted at one end thereof into the front surface securing holder of the Er:YAG laser beam-generating section;
a focusing lens inserted into an inside passage of the focusing lens holder so that the laser beam can be concentrated on a skin punch portion;
a connection device installed at the front end of the focusing lens holder; and
a buffer spring interposed between the connection device and the focusing lens holder.

13. A blood glucose meter comprising:
a minimum painless lancing device according to claim 1;
a connector to insert a blood glucose sensor strip;
a blood glucose measuring circuit; and
means to display measured blood glucose value.

14. A blood glucose meter comprising:
a minimum painless lancing device according to claim 2;
a connector to insert a blood glucose sensor strip;
a blood glucose measuring circuit; and
means to display measured blood glucose value.

15. A blood glucose meter comprising:
a minimum painless lancing device according to claim 3;
a connector to insert a blood glucose sensor strip;
a blood glucose measuring circuit; and
means to display measured blood glucose value.

16. A blood glucose meter comprising:
a minimum painless lancing device according to claim 4;
a connector to insert a blood glucose sensor strip;
a blood glucose measuring circuit; and
means to display measured blood glucose value.

17. The blood glucose meter according to claim 13, further including a program to record/manage results of the blood glucose measuring installed in a microprocessor unit (MPU).
